(19) United States
(12) Patent Application Publication
(10) Pub. No.: US 2005/0075627 A1
(43) Pub. Date: Apr. 7, 2005

(54) Antibiotic drug delivery system

(76) Inventor: Mervyn Simon Andrew Ward, Cheshire (GB)

Correspondence Address: Ropes & Gray, One International Place, Boston, MA, 02110-2624, US

(21) Appl. No.: 10/482,677

(22) PCT Filed: Jun. 26, 2002
(86) PCT No.: PCT/GB02/02929

(30) Foreign Application Priority Data
Jul. 2, 2001 (GB) 0116142.1

(51) Int. Cl. A61K 9/22

(52) U.S. Cl. 604/890.1

(57) ABSTRACT

An antibiotic drug delivery system for controlled infusion of an antibiotic drug to a patient, which system comprises (i) a delivery device for providing an infusion of the antibiotic at a controlled rate, together with (ii) a control system for varying the infusion rate and time of dosing of the antibiotic according to one or more parameters of the drug so as to maintain antibiotic levels in the patient of a desired percentage above the accepted MIC for that antibiotic.

### CROSS-REFERENCE TO RELATED APPLICATIONS

This application is a national stage filing under 35 U.S.C. 371 of International Application No. PCT/GB02/02929, filed Jun. 26, 2002, which claims priority from United Kingdom Patent Application No. 0116142.1, filed Jul. 2, 2001 the specifications of which are incorporated by reference herein. International Application No. PCT/GB02/02929 was published under PCT Article 21(2) in English.

The invention relates to the field of antibacterial agents. In particular it relates to improvements in the infusion of antibiotic drugs and apparatus for providing controlled infusion of such drugs.

The dosing of antibiotics is historically based on three factors:

a) The drug's half life dictating once every 24 hrs (q24); every 12 hours (q12); every 8 hours (q8) or every 6 hours (q6).

b) The dose selected, chosen by using the Minimum Inhibitory Concentration (MIC) for the range of bacteria the antibiotic is effective against and then taking the ‘breakpoint’ MIC for the majority of organisms and dosing to achieve this concentration level.

c) Tolerability at the various dosing levels is taken into account.

Since the majority of antibiotics gained regulatory approval, several authors have confirmed that pharmacokinetically the correct dosing targets for several classes of antibiotics are to achieve drug levels above the MIC for a certain percentage of the dosing interval. This percentage value is variable for different drug classes e.g. carbapenems are 40% and penicillins and cephalosporins are between 50% and 60% of the time. This rule is seen as being general for that class of drug and has been illustrated by several workers including Vogelman B et al. J Infect Dis 1988; 158:831-847; Am J Med 77 (suppl 6A):43; R. Walker, D. Andes, R. Conklin, S. Ebert W. Craig; Clin Infec Dis 1998; Craig W A et al.

Additional work has shown that improved results can be achieved by using longer infusions instead of intermittent dosing. Thus 1-3 hour infusions, instead of bolus dosing or infusions under 30 minutes, may achieve better microbiological results, dependent upon the bacterium and antibacterial agent.

At present, delivery of antibiotics at the bedside is often via an infusion bag dripped into a patient over a 30 minute time period or via bolus injection from a syringe over a shorter time period. Thus the whole process does not take account of the pharmacokinetic principle and may result in overdosing to ensure efficacy. That is to say if one dose is applied to all MIC's, it may result in overdosing for some organisms and under dosing for others.

In summary, current methods for delivering continuous infusions may result for example in tolerance problems, overdosing, underdosing, and the development of antibiotic resistance.

The invention is based at least in part on the realisation that pharmacokinetic or pharmacodynamic data for a particular antibiotic drug can be used to derive infusion characteristics for that drug which can be programmed into a delivery system to provide controlled infusion of that particular drug. We believe that a delivery system that uses such infusion characteristics may be able to provide the best available administration regime for that particular drug. We anticipate that use of the system will mean less antibiotic is required per therapeutic treatment and that treatment times will be shorter.

Therefore in a first aspect of the present invention we provide an antibiotic drug delivery system for controlled infusion of an antibiotic drug to a patient, which system comprises

(i) a delivery device for providing an infusion of the antibiotic at a controlled rate, together with

(ii) a control system for varying the infusion rate and time of dosing of the antibiotic according to one or more parameters of the drug so as to maintain antibiotic levels in the patient of a desired percentage above the accepted MIC for that antibiotic.

The parameters of the drug include, without limitation, pharmcokinetic and pharmacodynamic parameters as well as the established MIC for the drug.

It will be understood that the delivery device for providing controlled infusion of the antibiotic include conventional devices such as pumps, syringes and control valves. In general terms, the antibiotic drug is kept in a reservoir such as a bag, vial or syringe and then pumped or gravity fed. Often the device will comprise two main elements such as for example a reservoir and pump or alternatively a syringe and mechanical means acting on the syringe plunger and/or barrel.

Particularly useful devices include pumps used for target controlled infusion in other technical fields such as the Diprifusor™ pump used for delivering the anaesthetic Diprivan (propofol). See for example U.S. Pat. Nos. 5,882,338 and 6,019,745, also PCT/GB94/00909.

The control system for varying the infusion rate of the antibiotic according to one or more pharmacokinetic parameters so as to maintain antibiotic levels in the patient of a desired percentage above the accepted MIC for that antibiotic, may comprise any convenient mechanical and/or electrical elements. The relevant instructions for the control system may for example be provided via an optical recognition system eg. barcodes/scanner or radiofrequency devices. In particular aspects of the invention the instructions are conveniently provided on any convenient data storage medium. Without limitation this may be a computer (such as a PC) or a computer chip holding the relevant mechanistic data eg. pharmacokinetic formula and MIC data for a given antibiotic/bacterium combination.

Such a computer chip represents a particular independent aspect of the invention. This may be attached or linked to any element of the delivery device, conveniently to the element containing the antibiotic drug. This allows the delivery device to be set up for a different antibiotic, if required, merely by changing the reservoir.

The control system for varying the infusion rate and time of dosing of the antibiotic according to one or more of its mechanistic parameters so as to maintain antibiotic levels in the patient of a desired percentage above the accepted MIC for that antibiotic represents a further and independent aspect of the invention.

The control system is conveniently programmed to reflect globally available mechanistic parameters and/or patient data for a given drug. This may be supplemented by local hospital data and/or local patient data. If desired, one or more of these may be entered manually at any time prior to treatment. By way of non-limiting example patient age, weight, renal status and other personal information may be entered immediately prior to treatment.

The control system may be set up to compare global and local data and to act accordingly. If required a manual override facility may be provided. It will be appreciated that such a facility must be used with caution.

In a further aspect of the invention the control system is provided with one or more failsafes. These can be used for example to ensure that proscribed maximum and minimum values are not exceeded and cannot be overridden.

Any convenient percentage about the MIC may be selected if appropriate for a given microorganism. Whilst we don't wish to be limited by theoretical considerations this could be up to 20, 25, 30, 35, 40, 45, 50, 55, 60, 65, 70, 75, 80, 85, 90, 95 or 100% above the MIC. Still further it could be more than 100%.

The drug delivery system of the invention may be used to deliver any convenient antibiotic drug. These include all carbapenems such as meropenem, imipenem, imipenem/cilastatin, ertapenem, banipenem, and in particular meropenem. Further antibiotics include penems such as faropenem; cephalosporins such as ceftriaxone, cefepime, and ceftazidime; penicillins such as ampicillin; oxazolidinones such as linezolid. The system may also be used to deliver drug combinations such as piperacillin/tazobactam.

The drug delivery system of the invention may be used to provide antibiotic treatment for all hospital in-patient and out-patient therapy indications.

The patient may be human or animal.

A further aspect of the invention relates to the use of the drug delivery system of the invention for the infusion of an antibiotic drug.

Another aspect of the invention relates to a method of treatment of the human or animal body using an antibiotic drug which method comprises the use of the drug delivery system of the invention.

The invention will now be illustrated, but not limited, by reference to the following Figures wherein:

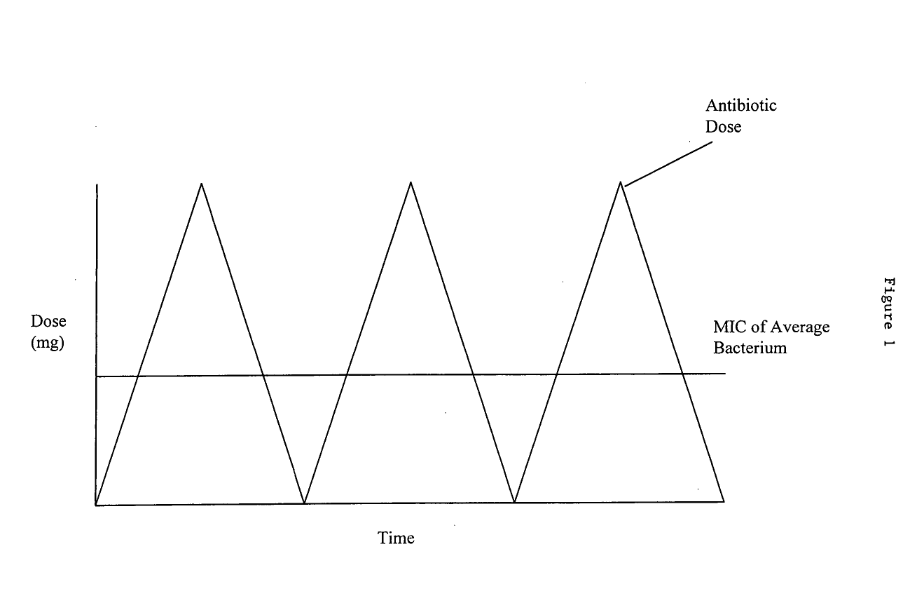

FIG. 1 shows current dosing strategy and the likely antibiotic concentration in a patient measured against time in comparison with the MIC of an average bacterium.

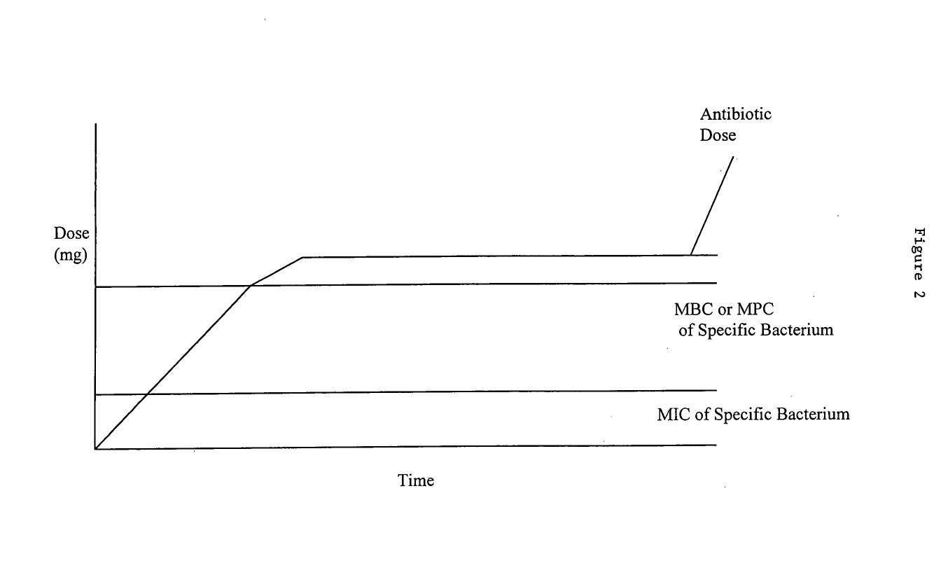

FIG. 2 shows the controlled antibiotic dose above the MIC of a specific bacterium as may be established in a patient using the drug delivery system of the invention.

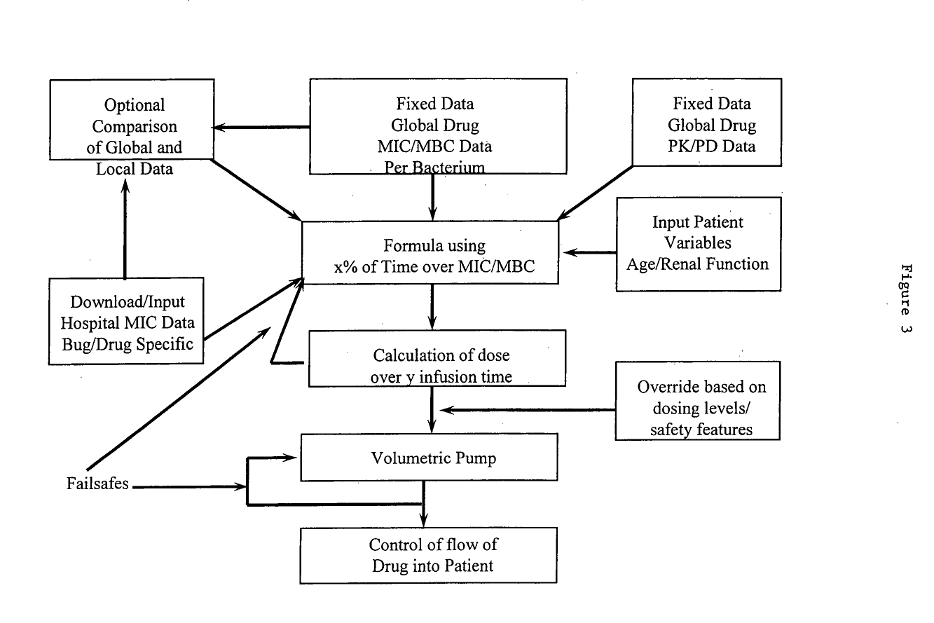

FIG. 3 illustrates elements of a control system for varying the infusion rate and time of dosing.

1. An antibiotic drug delivery system for controlled infusion of an antibiotic drug to a patient, which system comprises
(i) a delivery device for providing an infusion of the antibiotic at a controlled rate, together with
(ii) a control system for varying the infusion rate and time of dosing of the antibiotic according to one or more parameters of the drug so as to maintain antibiotic levels in the patient of a desired percentage above the accepted MIC for that antibiotic.

2. An antibiotic drug delivery system as claimed in claim 1, wherein the delivery device comprises a reservoir and pump.

3. An antibiotic drug delivery system as claimed in claim 1, wherein the delivery device comprises a syringe and mechanical means acting on the syringe plunger and/or barrel.

4. An antibiotic drug delivery system as claimed in claim 1, wherein the control system includes instructions provided on a data storage medium.

5. An antibiotic drug delivery system as claimed in claim 4, wherein the data storage medium is a computer chip.

6. An antibiotic drug delivery system as claimed in claim 4, wherein the data storage medium is a computer.

7. An antibiotic drug delivery system as claimed in claim 1, wherein the control system includes mechanistic data for one or more antibiotic/bacterium combinations.

8. An antibiotic drug delivery system as claimed in claim 1, wherein the control system includes local mechanistic and/or patient data for one or more antibiotic/bacterium combinations.

9. An antibiotic drug delivery system as claimed in claim 1, wherein the control system includes global mechanistic and/or patient data for one or more antibiotic/bacterium combinations.

10. An antibiotic drug delivery system as claimed in claim 1, wherein the control system is set up to compare local and global mechanistic and/or patient data for one or more antibiotic/bacterium combinations.

11. An antibiotic drug delivery system as claimed in claim 1, further comprising a manual override facility.

12. An antibiotic drug delivery system as claimed in claim 1, further comprising one or more failsafes.

13. A computer chip comprising instructions system for varying the infusion rate and time of dosing of an antibiotic according to one or more parameters of the drug so as to maintain antibiotic levels in a patient of a desired percentage above the accepted MIC for that antibiotic.

14. A control system for varying the infusion rate and time of dosing of an antibiotic according to one or more parameters of the drug so as to maintain antibiotic levels in a patient of a desired percentage above the accepted MIC for that antibiotic.

15. An antibiotic drug delivery system as claimed in claim 1, wherein the antibiotic drug is a carbapenem antibiotic.

16. An antibiotic drug delivery system as claimed in claim 1, wherein the antibiotic drug is a penem antibiotic.

17. An antibiotic drug delivery system as claimed in claim 1, wherein the antibiotic drug is an oxazolidinone antibiotic.
